(19) United States
(12) Patent Application Publication
(10) Pub. No.: US 2014/0328972 A1
(43) Pub. Date: Nov. 6, 2014

(54) Xylanases for Animal Feed

(71) Applicant: Novozymes A/S, Bagsvaerd (DK)

(72) Inventors: Morten Fischer, Copenhagen (DK); Dan Pettersson, Lynge (DK)

(21) Appl. No.: 14/335,245

(22) Filed: Jul. 18, 2014

Related U.S. Application Data
(63) Continuation of application No. 12/441,627, filed on Mar. 17, 2009, now abandoned.
(60) Provisional application No. 60/848,717, filed on Oct. 2, 2006.

(30) Foreign Application Priority Data
Sep. 29, 2006 (DK) PA 2006 01262

(51) Int. Cl. A23K 1/165; C12N 9/24

(52) U.S. Cl. CPC A23K 1/1653; C12N 9/248
USPC 426/2; 426/61

(57) ABSTRACT

The present invention relates to the use in animal feed of a xylanase having a percentage of identity to a Paenibacillus xylanase having the sequence of amino acids 1-184 of SEQ ID NO: 4 of at least 82.7%, as well as to feed additives and feed compositions comprising such xylanase. These xylanases are significantly better than known animal feed xylanases to solubilize and also degrade insoluble fibre polysaccharides (Non-Starch Polysaccharides, abbreviated NSP), such as arabinoxylan polysaccharides.

### CROSS-REFERENCE TO RELATED APPLICATIONS

This application is a continuation of U.S. application Ser. No. 12/441,627 filed on Mar. 17, 2009, now pending, which is a 35 U.S.C. 371 national application of PCT/EP2007/060242 filed Sep. 27, 2007, which claims priority or the benefit under 35 U.S.C. 119 of Danish application no. PA 2006 01262 filed Sep. 29, 2006 and U.S. provisional application No. 60/848,717 filed Oct. 2, 2006 the contents of which are fully incorporated herein by reference.

### REFERENCE TO SEQUENCE LISTING

This application contains a Sequence Listing in computer readable form, which is incorporated herein by reference.

### BACKGROUND OF THE INVENTION

1. Field of the Invention

This invention relates to the field of animal feed.

Cereal grains are important components of animal feed. They contain, i.a., plant polysaccharides which can function as a major nutritional component in the diet (e.g. starch), but also a number of non-starch polysaccharides (NSP) including, among others, arabinoxylans. Major farm animals like poultry and pigs lack the relevant enzymes in their digestive tracts for digesting the NSP. It is known in the art to use xylanases in animal feed in order to improve the feed utilization.

The present invention relates to the use in animal feed of a xylanase having a percentage of identity to a xylanase from Paenibacillus (amino acids 1-184 of SEQ ID NO: 4) of at least 82.7%.

2. Description of the Related Art

WO 2006/083240 discloses, in Example 6, the use in chicken feed of a xylanase designated XylA1A. The amino acid sequence of XylA1A corresponds to SEQ ID NO: 2 in WO 2006/083240, which is identical to the mature part of UNIPROT:Q6TLP3 which is included in the present sequence listing as SEQ ID NO: 9. According to the UNIPROT database entry this sequence derives from a bacterium isolated from an environmental sample. The percentage identity of the xylanase of SEQ ID NO: 9 to amino acids 1-184 of SEQ ID NO: 4 is below 82.7%.

The RONOZYME WX xylanase is a known mono-component animal feed xylanase derived from Thermomyces lanuginosus and commercially available from DSM Nutritional Products, Wurmisweg 576, CH-4303 Kaiseraugst, Switzerland. This xylanase and its use in animal feed are also described in WO 96/23062. This xylanase does not have a molecular weight below 24 kDa.

A xylanase from Paenibacillus pabuli having the amino acid sequence of amino acids 1-182 of SEQ ID NO: 2 herein, and its use in a process for preparing a dough-based product, are described in WO 2005/079585.

The amino acid sequence of a xylanase from Paenibacillus sp. KCTC 8848P was submitted to the public Uniprot database with accession no. UNIPROT:Q9F9B9, and is included in the present sequence listing as SEQ ID NO: 4.

WO 97/13853 discloses (SEQ ID NO: 6) a xylanase from Aspergillus niger which is identical except for one amino acid to the sequence of amino acids 1-188 of SEQ ID NO: 6 herein (“xyl II” from Aspergillus niger).

WO 2004/018662 discloses (as SEQ ID NO: 9 in WO 2004/018662) another xylanase from Aspergillus niger which is identical to SEQ ID NO: 8 herein (“xyl III” from Aspergillus niger).

Chesson et al., 1997, J. Sci. Food Agric. 75: 289-295, report studies of cell wall porosity and available surface area of wheat straw and wheat grain fractions.

It is an object of the present invention to improve the solubilization and/or degradation of insoluble non-starch polysaccharides (NSP) such as arabinoxylans with a view to improving the nutritional value of animal feed, e.g. by improving the feed conversion ratio (FCR), the growth rate, and/or the weight gain.

### SUMMARY OF THE INVENTION

The present invention relates to the use in animal feed of a xylanase having a percentage of identity to amino acids 1-184 of SEQ ID NO: 4 of at least 82.7%, the percentage of identity being determined by i) aligning the two amino acid sequences using the Needle program, with the BLOSUM62 substitution matrix, a gap opening penalty of 10, and a gap extension penalty of 0.5; ii) counting the number of exact matches in the alignment; iii) dividing the number of exact matches by the length of the shortest of the two amino acid sequences, and iv) converting the result of the division of iii) into percentage.

The invention also relates to the use of such xylanase in the preparation of a composition for use in animal feed.

The invention furthermore relates to a composition comprising such xylanase and (a) at least one fat soluble vitamin; (b) at least one water soluble vitamin; and/or (c) at least one trace mineral.

Still further, the invention relates to an animal feed composition having a crude protein content of 50 to 800 g/kg and comprising such xylanase, as well as a method for improving the nutritional value of an animal feed, wherein such xylanase is added to the feed.

Finally, the invention relates to the use of such xylanase for the solubilization and/or degradation of non-starch polysaccharides during gastric and intestinal digestion; and for pre-treatment of animal feed or animal feed components.

### DETAILED DESCRIPTION OF THE INVENTION

In what follows, the expression “xylanase of the invention” refers to a xylanase for use according to the invention, as described herein.

### EC Classes of Enzymes—Bernard Henrissat Glycoside Hydrolase Families

Enzymes can be classified on the basis of the handbook Enzyme Nomenclature from NC-IUBMB, 1992), see also the ENZYME site at the internet: http://www.expasy.ch/enzyme/. ENZYME is a repository of information relative to the nomenclature of enzymes. It is primarily based on the recommendations of the Nomenclature Committee of the International Union of Biochemistry and Molecular Biology (IUB-MB) and it describes each type of characterized enzyme for which an EC (Enzyme Commission) number has been provided (Bairoch A. The ENZYME database, 2000, Nucleic Acids Res 28:304-305). This IUB-MB Enzyme nomenclature is based on their substrate specificity and occasionally on their molecular mechanism; such a classification does not reflect the structural features of these enzymes.

Another classification of certain glycoside hydrolase enzymes, such as endoglucanase, xylanase, galactanase, mannanase, dextranase and alpha-galactosidase, in families based on amino acid sequence similarities has been proposed a few years ago. They currently fall into 90 different families: See the CAZy(ModO) internet site (Coutinho, P. M. & Henrissat, B. (1999) Carbohydrate-Active Enzymes server at: http://afmb.cnrs-mrs.fr˜cazy/CAZY/index.html (corresponding papers: Coutinho, P. M. & Henrissat, B. (1999) Carbohydrate-active enzymes: an integrated database approach. In “Recent Advances in Carbohydrate Bioengineering”, H. J. Gilbert, G. Davies, B. Henrissat and B. Svensson eds., The Royal Society of Chemistry, Cambridge, pp. 3-12; Coutinho, P. M. & Henrissat, B. (1999) The modular structure of cellulases and other carbohydrate-active enzymes: an integrated database approach. In “Genetics, Biochemistry and Ecology of Cellulose Degradation”., K. Ohmiya, K. Hayashi, K. Sakka, Y. Kobayashi, S. Karita and T. Kimura eds., Uni Publishers Co., Tokyo, pp. 15-23).

### Xylanase

For the present purposes, a xylanase means a protein, or a polypeptide, having xylanase activity.

Xylanase activity can be measured using any assay, in which a substrate is employed, that includes 1,4-beta-D-xylosidic endo-linkages in xylans. Assay-pH and assay-temperature are to be adapted to the xylanase in question. Examples of assay-pH-values are pH 4, 5, 6, 7, 8, 9, 10, or 11. Examples of assay-temperatures are 30, 35, 37, 40, 45, 50, 55, 60, 65, 70 or 80° C.

Different types of substrates are available for the determination of xylanase activity e.g. Xylazyme cross-linked arabinoxylan tablets (from MegaZyme), or insoluble powder dispersions and solutions of azo-dyed arabinoxylan.

For assaying xylanase in feed, premix and the like samples, the enzyme is extracted at temperatures ranging from 50° C. up to 70° C. (with the higher temperatures used for the more thermostable enzymes) in an extraction medium typically consisting of a phosphate buffer (0.1 M and a pH adjusted to the pH optima of the enzyme in question) for a time period of 30 to 60 min. A preferred xylanase assay is disclosed in Example 4.

All measurements are based on spectrophotometric determination principles at approx. 590-600 nm. The enzyme, or the extracted enzyme, as applicable, is incubated with a known amount of substrate and the colour release is measured relative to a standard curve obtained by adding known amounts of an enzyme standard to a similar control diet without enzyme.

When no control feed is available, a known amount of enzyme is added to the sample (spiking) and from the differences in response between spiked and non-spiked sample the added amount of enzyme can be calculated.

In a particular embodiment, the xylanase is an enzyme classified as EC 3.2.1.8 (see the ENZYME site referred to above). The official name is endo-1,4-beta-xylanase. The systematic name is 1,4-beta-D-xylan xylanohydrolase. Other names may be used, such as endo-(1-4)-beta-xylanase; (1-4)-beta-xylan 4-xylanohydrolase; endo-1,4-xylanase; xylanase; beta-1,4-xylanase; endo-1,4-xylanase; endo-beta-1,4-xylanase; endo-1,4-beta-D-xylanase; 1,4-beta-xylan xylanohydrolase; beta-xylanase; beta-1,4-xylan xylanohydrolase; endo-1,4-beta-xylanase; beta-D-xylanase. The reaction catalyzed is the endohydrolysis of 1,4-beta-D-xylosidic linkages in xylans.

According to the CAZy(ModO) site referred to above, xylanases are presently classified in either of the following Glycoside Hydrolyase Families: 5, 8, 10, 11, 16, 43, or 62. E.g., GH Family 11 glycoside hydrolases can be characterized as follows:
CAZy Family: Glycoside Hydrolase Family 11
Known Activities: Xylanase (EC 3.2.1.8)
Mechanism: Retaining
Catalytic Nucleophile/Base: Glu (experimental)
Catalytic Proton Donor: Glu (experimental)
3D Structure Status: Available (see PDB)
Fold: Beta-jelly roll
Clan: GH-C

In particular embodiments, the xylanase of the invention is i) a xylanase of Glycoside Hydrolyase (GH) Family 5, 8, 10, 11, 16, 43, or 62, preferably of GH Family 10, or 11, more preferably of GH Family 11. The expression “of Glycoside Hydrolase Family NN” means that the xylanase in question is or can be classified in GH family “NN” (e.g. 10, or 11).

In another particular embodiment, the xylanase of the invention is derived from a bacterial xylanase, preferably from a bacterium of (i) the phylum of Firmicutes; (ii) the class of Bacilli; (iii) the order of Bacillales; (iv) the family of Paenibacillaceae; or (v) the genus of Paenibacillus; even more preferably from a bacterium of (vi) the species of Paenibacillus pabuli, Paenibacillus polymyxa, or Paenibacillus sp.; most preferably from (vii) strains of Paenibacillus pabuli, or Paenibacillus polymyxa.

The expression “xylanase derived from a bacterial (or Firmicutes, . . . , or Paenibacillus pabuli) xylanase” as used hereinabove includes any wild-type xylanase isolated from the bacterium in question, as well as variants or fragments thereof which retain xylanase activity.

The term “variant” refers to a xylanase which comprises a substitution, deletion, and/or insertion of one or more amino acids as compared to the specified xylanase. The variant may be a natural variant (allelic variant), or prepared synthetically. Preferably, amino acid changes are of a minor nature, e.g., conservative amino acid substitutions that do not significantly affect the folding and/or activity of the protein; small deletions; small amino- or carboxyl-terminal extensions, such as an amino-terminal methionine residue; a small linker peptide; or a small extension that facilitates purification by changing net charge or another function, such as a poly-histidine tract, an antigenic epitope or a binding domain.

A “fragment” of a specified xylanase has one or more amino acids deleted from the amino and/or carboxyl terminus of the amino acid sequence of the xylanase.

For purposes of the above definitions of variant and fragment, the term “small” as well as the term “one or more” refer to a maximum of 30 changes as compared to the specified xylanase. In preferred embodiments of either of these definitions, the number of changes is below 30, 25, 20, 15, 10, or below 5.

Examples of conservative substitutions are within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly as well as these in reverse.

In addition to the 20 standard amino acids, non-standard amino acids (such as 4-hydroxyproline, 6-N-methyl lysine, 2-aminoisobutyric acid, isovaline, and alpha-methyl serine) may be substituted for amino acid residues of a wild-type polypeptide. A limited number of non-conservative amino acids, amino acids that are not encoded by the genetic code, and unnatural amino acids may be substituted for amino acid residues. “Unnatural amino acids” have been modified after protein synthesis, and/or have a chemical structure in their side chain(s) different from that of the standard amino acids. Unnatural amino acids can be chemically synthesized, and preferably, are commercially available, and include pipecolic acid, thiazolidine carboxylic acid, dehydroproline, 3- and 4-methylproline, and 3,3-dimethylproline.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for biological activity (i.e., xylanase activity) to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309:59-64. The identities of essential amino acids can also be inferred from analysis of identities with polypeptides which are related to a polypeptide according to the invention.

Single or multiple amino acid substitutions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochem. 30:10832-10837; U.S. Pat. No. 5,223,409; WO 92/06204), and region-directed mutagenesis (Derbyshire et al., 1986, Gene 46:145; Ner et al., 1988, DNA 7:127).

Mutagenesis/shuffling methods can be combined with high-throughput, automated screening methods to detect activity of cloned, mutagenized polypeptides expressed by host cells. Mutagenized DNA molecules that encode active polypeptides can be recovered from the host cells and rapidly sequenced using standard methods in the art. These methods allow the rapid determination of the importance of individual amino acid residues in a polypeptide of interest, and can be applied to polypeptides of unknown structure.

In a further particular embodiment the xylanase of the invention is derived from a fungal xylanase. The above definition of “derived from” (in the context of bacterial xylanases) is applicable by analogy also to fungal xylanases. Fungal xylanases include yeast and filamentous fungal xylanases. In preferred embodiments, the xylanase is derived from a fungus of (i) the phylum of Ascomycota; (ii) the class of Pezizomycotina; (iii) the order of Eurotiomycetes; (iv) the sub-order of Eurotiales; (v) the family of Trichocomaceae, preferably the mitosporic Trichocomaceae; even more preferably from a fungus of (vi) the genus Aspergillus; most preferably from (vii) strains of Aspergillus niger. It will be understood that the definition of the aforementioned species includes both the perfect and imperfect states, and other taxonomic equivalents e.g., anamorphs, regardless of the species name by which they are known. Those skilled in the art will readily recognize the identity of appropriate equivalents.

Strains of the abovementioned bacteria and fungi are readily accessible to the public in a number of culture collections, such as the American Type Culture Collection (ATCC), Deutsche Sammlung von Mikroorganismen and Zellkulturen GmbH (DSMZ), Centraalbureau Voor Schimmelcultures (CBS), and Agricultural Research Service Patent Culture Collection, Northern Regional Research Center (NRRL).

Questions relating to taxonomy can be solved by consulting a taxonomy data base, such as the NCBI Taxonomy Browser which is available at the following internet site: http://www.ncbi.nlm.nih.gov/Taxonomy/taxonomyhome.html/. However, preferably reference is had to the following handbooks: Dictionary of the Fungi, 9^{th} edition, edited by Kirk, P. M., P. F. Cannon, J. C. David & J. A. Stalpers, CAB Publishing, 2001; and Bergey's Manual of Systematic Bacteriology, Second edition (2005).

The term “a” as used herein in whatever context means “one or more”, preferably “at least one”. This is the case, e.g., for the use in claim 1 of “a” xylanase as specified, which is considered equivalent to claiming the use of “at least one” or “one or more” such xylanases.

The term a “mature” polypeptide or mature amino acid sequence refers to that part of an amino acid sequence which remains after a potential signal peptide part and a potential propeptide part have been cleaved off. Some variation may be observed in the mature parts of enzymes, depending on i.a. expression hosts and fermentation conditions. E.g., experience shows that sometimes also minor C-terminal truncations occur during the secretion process. The term mature part as used herein also takes into account such C-terminal truncations, if any. While the mature peptide part may be identified by computer programs known in the art (e.g. SignalP 3.0, see J. D. Bendtsen et al, J. Mol. Biol., 340:783-795, 2004), preferably it is identified by determination of the N-terminal, and preferably also C-terminal, of the expressed and secreted, if relevant, xylanase enzyme. E.g., according to our observations, the mature part of the xylanase of SEQ ID NO: 2 is amino acids 1-182 thereof, and the mature part of the xylanase of SEQ ID NO: 4 is amino acids 1-184 thereof.

In a particular embodiment the xylanase of the invention is isolated, i.e. essentially free of other polypeptides of enzyme activity, e.g., at least about 20% pure, preferably at least about 40% pure, more preferably about 60% pure, even more preferably about 80% pure, most preferably about 90% pure, and even most preferably about 95% pure, as determined by SDS-PAGE. As it is generally known in the art, for detection purposes the SDS-gel can be stained with Coomassie or silver staining. It should be ensured that overloading has not occurred, e.g. by checking linearity by applying various concentrations in different lanes on the gel. Such polypeptide preparations are in particular obtainable using recombinant methods of production, whereas they are not so easily obtained and also subject to a much higher batch-to-batch variation when the polypeptide is produced by traditional fermentation methods.

The polypeptides comprised in the composition of the invention are preferably also purified. The term purified refers to a protein-enriched preparation, in which a substantial amount of low molecular components, typical residual nutrients and minerals originating from the fermentation, have been removed. Such purification can e.g. be by conventional chromatographic methods such as ion-exchange chromatography, hydrophobic interaction chromatography and size exclusion chromatography (see e.g. Protein Purification, Principles, High Resolution Methods, and Applications. Editors: Jan-Christer Janson, Lars Ryden, VCH Publishers, 1989). Example 2 of WO 2005/079585 describes a suitable procedure for the purification of the Paenibacillus pabuli xylanase, expressed from Bacillus subtilis.

The use of an isolated and/or purified polypeptide according to the invention is advantageous. For instance, it is much easier to correctly dose enzymes that are essentially free from interfering or contaminating other enzymes. The terms correctly dose refer in particular to the objective of obtaining consistent and constant animal feeding results, and the capability of optimizing dosage based upon the desired effect.

### Identity

The relatedness between two amino acid sequences is described by the parameter “identity”.

The present invention relates to the use in animal feed of a xylanase having a percentage of identity to amino acids 1-184 of SEQ ID NO: 4 of at least 82.7%

In particular embodiments, the degree of identity is at least 85%, 90%, 95%, 97%, or at least 99%. In additional embodiments, the degree of identity is at least 82.8%, 82.9%, 83.0%, 83.2%, 83.4%, 83.6%, 83.8%, 84.0%, 84.5%, 85.0%, or at least 85.5%. In still further embodiments, the degree of identity is at least 86%, 87%, 88%, 89%, 91%, 92%, 93%, 94%, 96%, or at least 98%.

The invention in particular relates to the use in animal feed of a xylanase having a percentage of identity to amino acids 1-182 of SEQ ID NO: 2 of at least 85%, and/or a percentage of identity to amino acids 1-184 of SEQ ID NO: 4 of at least 86%.

In still further particular embodiments, the xylanase of the invention comprises (preferably has, or consists of) a mature part of any one of the xylanases of SEQ ID NO: 2, SEQ ID NO: 4, SEQ ID NO: 6, and/or SEQ ID NO: 8; or a variant or fragment thereof that has xylanase activity.

For purposes of the present invention, the degree of identity between two amino acid sequences is determined on the basis of an alignment of the two amino acid sequences made by using the Needle program from the EMBOSS package (http://emboss.org) version 2.8.0. The Needle program implements the global alignment algorithm described in Needleman, S. B. and Wunsch, C. D. (1970) J. Mol. Biol. 48, 443-453. The substitution matrix used is BLOSUM62, gap opening penalty is 10, and gap extension penalty is 0.5.

The degree of identity between an amino acid sequence of the present invention (“invention sequence”; e.g. amino acids 1-184 of SEQ ID NO: 4 and a different amino acid sequence (“foreign sequence”) is calculated as the number of exact matches in an alignment of the two sequences, divided by the length of the “invention sequence” or the length of the “foreign sequence”, whichever is the shortest. The result is expressed in percent identity.

An exact match occurs when the “invention sequence” and the “foreign sequence” have identical amino acid residues in the same positions of the overlap (in the alignment example below this is represented by “|”). The length of a sequence is the number of amino acid residues in the sequence (e.g. the length of amino acids 1-184 of SEQ ID NO: 4 is 184).

In the purely hypothetical alignment example below, the overlap is the amino acid sequence “HTWGER-NL” of Sequence 1; or the amino acid sequence “HGWGEDANL” of Sequence 2. In the example a gap is indicated by a “-”.

Hypothetical alignment example:

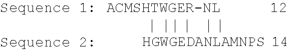

In this hypothetical example, the number of exact matches is 6. The length of the shortest sequence is 12. Accordingly the degree of identity of Sequence 1 to Sequence 2 is 50%.

In a particular embodiment, the percentage of identity of an amino acid sequence of a polypeptide with, or to, amino acids 1 to 184 of SEQ ID NO: 4 is determined by i) aligning the two amino acid sequences using the Needle program, with the BLOSUM62 substitution matrix, a gap opening penalty of 10, and a gap extension penalty of 0.5; ii) counting the number of exact matches in the alignment; iii) dividing the number of exact matches by the length of the shortest of the two amino acid sequences, and iv) converting the result of the division of iii) into percentage. The percentage of identity to, or with, other sequences of the invention such as amino acids 1-182 of SEQ ID NO: 2 is calculated in an analogous way.

### Animal Feed

The present invention is also directed to methods for using the xylanase of the invention in animal feed, as well as to feed compositions and feed additives comprising it.

The term animal includes all animals, including human beings. Examples of animals are non-ruminants, and ruminants. Ruminant animals include, for example, animals such as sheep, goat, and cattle, e.g. cow such as beef cattle and dairy cows. In a particular embodiment, the animal is a non-ruminant animal. Non-ruminant animals include mono-gastric animals, e.g. pig or swine (including, but not limited to, piglets, growing pigs, and sows); poultry such as turkeys, ducks and chickens (including but not limited to broiler chicks, layers); fish (including but not limited to salmon, trout, tilapia, catfish and carp); and crustaceans (including but not limited to shrimp and prawn).

In particular embodiments, the xylanase of the invention is for use in feed for (i) non-ruminant animals; preferably (ii) mono-gastric animals; more preferably (iii) pigs, poultry, fish, and crustaceans; or, most preferably, (iv) pigs and poultry.

The xylanase of the invention can be fed to the animal before, after, or simultaneously with the diet. The latter is preferred.

The term feed, feed composition, or diet means any compound, preparation, mixture, or composition suitable for, or intended for intake by an animal. More information about animal feed compositions is found below.

Cereal grains are important components of animal feed. Cereal grains contain plant polysaccharides, some of which, e.g. starch, can function as a major nutritional component of the diet. But cereal grains also contain various kinds of non-starch polysaccharides (NSP), which cannot be utilized by non-ruminant animals such as poultry and pigs.

Examples of NSP are xylans, arabinoxylans, beta-glucans, and cellulose. The type and amount of NSP vary from cereal to cereal. The following are examples of approximate NSP content (%, w/w, dry matter) of various cereal grains: Pearled rice 1%, sorghum 5%, maize 8%, wheat 11%, rye 13%, triticale 16%, and barley 17%. For wheat, triticale and maize, arabinoxylans make up more than 50% of the NSP, whereas for barley, sorghum, rye, and rice the arabinoxylans make up approximately 25-45% of the NSP, i.e. still a substantial amount.

For arabinoxylans and beta-glucans, a distinction is made between soluble and insoluble polysaccharides. The terms soluble and insoluble are known in the art and refer to water solubility/insolubility, in particular to the form (soluble/insoluble) of these polysaccharides a) under digestive conditions, b) under intestinal conditions (in the small intestine), or preferably c) after an in vitro procedure as outlined in Example 1 (i.e., 1.5 hours at pH 3.0 and 40° C. in the presence of pepsin, and 4.5 hours at pH 6.8 and 40° C. in the presence of pancreatin).

Insoluble arabinoxylans are associated with the encapsulation of nutrients such as starch and protein. This encapsulation allows valuable nutrients to by-pass the digestion. When insoluble arabinoxylans are also digested, or solubilized, an improved exposure of nutrients results.

In a particular embodiment, the xylanase of the invention is capable of solubilizing insoluble fibre polysaccharides, such as NSP. Accordingly, the invention relates to the use of a xylanase of the invention for the solubilization of (otherwise insoluble) non-starch polysaccharides during gastric and intestinal digestion. Preferred non-starch polysaccharides are arabinoxylans (arabinoxylan polysaccharides).

The term polysaccharide is known in the art to designate saccharides with 10 or more monosaccharides (see e.g. Food Chemistry, 3^{rd} edition, Springer Verlag, ISBN 3-540-40817-7, Belitz, Grosch, Schieberle (editors), section 4.3.1 on p. 294), in other words, with a degree of polymerization (DP) of at least 10.

Polysaccharides with a DP of at least 10 can be distinguished from oligosaccharides with a DP below 10 as is known in the art, e.g. by Gel filtration on Biogel P-2 in supernatants obtained after 80% ethanol precipitation (see “The Uppsala method for rapid analysis of total dietary fiber” by Theander et al, in particular FIG. 2 on p. 277, in New Developments in Dietary Fiber, Furda and Brine (editors), Plenum Press, 1990, p. 273-281).

In particular, the xylanase of the invention it is capable of reducing the amount of insoluble xylans and arabinoxylans in an in vitro model mimicking the gastric and small intestinal digestion steps in monogastric digestion—as described in Examples 1, 2, and 5 herein. Preferably, the amount of residual (i.e., after incubation with xylanase) insoluble arabinoxylans is not higher than 85% (w/w), more preferably not higher than 84, 83, 82, 81, 80, 79, 78, 77, 76, 75, 74, 73, 72, 71, or 70% (w/w) relative to a control without added xylanase enzyme (100%). This corresponds to a reduction of the amount of insoluble arabinoxylans of at least 15%, preferably at least 16, 17, 18, 19, 20, 21, 22, 23, 24, 25, 26, 27, 28, 29, or at least 30% (w/w) relative to a control without added xylanase enzyme (0%).

In still further particular embodiments, the conditions of the in vitro model are: (i) substrate (diet): 0.35 g wheat, 0.21 g barley, 0.13 g soy bean meal, and 0.11 g wheat bran, provided as a premixed diet, milled to pass a 0.5 mm screen; (ii) a gastric step incubation in which the diet is incubated with 0.1 ml of the xylanase to be tested together with 4.1 ml 0.072 M HCl for 1.5 hours and with 0.5 ml 0.072 M HCl/pepsin (Sigma P-7000; 3000 U/g diet) for 1 hour (i.e. 30 min HCl-substrate premixing) at pH 3.0 and 40° C.; (iii) a subsequent intestinal step incubation with 0.9 ml 0.215M NaOH plus 0.4 ml 1M NaHCO₃ and pancreatin 8 mg/g diet for 4 hours (Sigma P-7545) at pH 6.8-7.0 and 40° C.; followed by (iv) a determination of the amount of residual insoluble arabinoxylan, e.g. using the Uppsala method, as described in Examples 1 and 5.

The invention also relates to the use of a xylanase of the invention for the degradation of non-starch polysaccharides during gastric and intestinal digestion. Preferred non-starch polysaccharides are fiber polysaccharides, in particular arabinoxylans (arabinoxylan polysaccharides). In particular, the xylanase of the invention it capable of degrading xylan and arabinoxylan polysaccharides in an in vitro model mimicking the gastric and small intestinal digestion steps in monogastric digestion—as described in Example 5 herein. Preferably, the amount of residual (i.e., after incubation with xylanase) total arabinoxylans is not higher than 94% (w/w), more preferably not higher than 93, 92, 91, 90, 89, 88, 87, 86, 85, 84, 83, 82, 81, or 80% (w/w) relative to a control without added xylanase enzyme (100%).

In still further particular embodiments, the conditions of the in vitro model are: (i) substrate (diet): 0.35 g wheat, 0.21 g barley, 0.13 g soy bean meal, and 0.11 g wheat bran, provided as a premixed diet, milled to pass a 0.5 mm screen; (ii) a gastric step incubation in which the diet is incubated with 0.1 ml of the xylanase to be tested together with 4.1 ml 0.072 M HCl for 1.5 hours and with 0.5 ml 0.072 M HCl/pepsin (Sigma P-7000; 3000 U/g diet) for 1 hour (i.e. 30 min HCl-substrate premixing) at pH 3.0 and 40° C.; (iii) a subsequent intestinal step incubation with 0.9 ml 0.215M NaOH plus 0.4 ml 1M NaHCO₃ and pancreatin 8 mg/g diet for 4 hours (Sigma P-7545) at pH 6.8-7.0 and 40° C.; followed by (iv) a determination of the amount of residual total arabinoxylan, e.g. using the Uppsala method, as described in Example 5.

The dosage of the xylanase of the invention can be optimized using simple trial-and-error methods as is known in the art. Different xylanases may have different optimum dosage ranges. Examples of suitable dosage ranges are: 0.1-500 mg enzyme protein (EP)/kg diet (substrate); preferably 0.2-400, 0.3-300, 0.4-200, or 0.5-100 mg EP/kg diet. Other preferred dosage ranges are 0.6-90, 0.7-80, 0.7-70, 1-70, 2-70, 3-70, 4-70, 5-70, 6-70, or 7-70 - all in mg EP/kg diet. Still further preferred enzyme dosages are from 10-500, 10-400, 10-300, 10-200, 10-100, 10-90, 10-80, or 10-70 - all in mg EP/kg diet. The amount of xylanase enzyme protein (EP) may be determined as described in Example 1.

For determining mg xylanase enzyme protein per kg feed, the xylanase is purified from the feed composition, and the specific activity of the purified xylanase is determined using a relevant assay. The xylanase activity of the feed composition as such is also determined using the same assay, and on the basis of these two determinations, the dosage in mg xylanase enzyme protein per kg feed is calculated.

The same principles apply for determining mg xylanase enzyme protein in feed additives. Of course, if a sample is available of the xylanase used for preparing the feed additive or the feed, the specific activity is determined from this sample (no need to purify the xylanase from the feed composition or the additive).

The term improving the nutritional value of an animal feed means improving the availability of nutrients, whereby the growth rate, weight gain, and/or feed conversion (i.e. the weight of ingested feed relative to weight gain) of the animal is/are improved.

The xylanase can be added to the feed in any form, be it as a purified and/or isolated xylanase, or in admixture with other components intended for addition to animal feed, i.e. in the form of animal feed additives, such as the so-called pre-mixes for animal feed.

In a further aspect the present invention relates to compositions for use in animal feed, such as animal feed, and animal feed additives, e.g. premixes.

Apart from the xylanase of the invention, the animal feed additives of the invention contain at least one fat-soluble vitamin, and/or at least one water soluble vitamin, and/or at least one trace mineral. Macro-minerals are also usually included in feed additives.

Further, optional, feed-additive ingredients are colouring agents, e.g. carotenoids such as beta-carotene, astaxanthin, and lutein; aroma compounds; stabilisers; antimicrobial peptides; polyunsaturated fatty acids; reactive oxygen generating species; and/or at least one other enzyme selected from amongst another xylanase (EC 3.2.1.8); and/or beta-glucanase (EC 3.2.1.4 or EC 3.2.1.6).

Examples of antimicrobial peptides (AMP's) are CAP18, Leucocin A, Tritrpticin, Protegrin-1, Thanatin, Defensin, Lactoferrin, Lactoferricin, and Ovispirin such as Novispirin (Robert Lehrer, 2000), Plectasins, and Statins, including the compounds and polypeptides disclosed in WO 03/044049 and WO 03/048148, as well as variants or fragments of the above that retain antimicrobial activity.

Examples of antifungal polypeptides (AFP's) are the Aspergillus giganteus, and Aspergillus niger peptides, as well as variants and fragments thereof which retain antifungal activity, as disclosed in WO 94/01459 and WO 02/090384.

Examples of polyunsaturated fatty acids are C18, C20 and C22 polyunsaturated fatty acids, such as arachidonic acid, docosohexaenoic acid, eicosapentaenoic acid and gamma-linoleic acid.

Examples of reactive oxygen generating species are chemicals such as perborate, persulphate, or percarbonate; and enzymes such as an oxidase, an oxygenase or a syntethase.

Usally fat- and water-soluble vitamins, as well as trace minerals form part of a so-called premix intended for addition to the feed, whereas macro minerals are usually separately added to the feed. A premix enriched with a xylanase of the invention is an example of an animal feed additive of the invention.

In a particular embodiment, the animal feed additive of the invention is intended for being included (or prescribed as having to be included) in animal diets or feed at levels of 0.01 to 10.0%; more particularly 0.05 to 5.0%; or 0.2 to 1.0% (% meaning g additive per 100 g feed). This is so in particular for premixes.

The following are non-exclusive lists of examples of these components:

Examples of fat-soluble vitamins are vitamin A, vitamin D3, vitamin E, and vitamin K, e.g. vitamin K3.

Examples of water-soluble vitamins are vitamin B12, biotin and choline, vitamin B1, vitamin B2, vitamin B6, niacin, folic acid and panthothenate, e.g. Ca-D-panthothenate.

Examples of trace minerals are manganese, zinc, iron, copper, iodine, selenium, and cobalt.

Examples of macro minerals are calcium, phosphorus and sodium.

The nutritional requirements of these components (exemplified with poultry and piglets/pigs) are listed in Table A of WO 01/58275. Nutritional requirement means that these components should be provided in the diet in the concentrations indicated.

In the alternative, the animal feed additive of the invention comprises at least one of the individual components specified in Table A of WO 01/58275. At least one means either of, one or more of, one, or two, or three, or four and so forth up to all thirteen, or up to all fifteen individual components. More specifically, this at least one individual component is included in the additive of the invention in such an amount as to provide an in-feed-concentration within the range indicated in column four, or column five, or column six of Table A.

The present invention also relates to animal feed compositions. Animal feed compositions or diets have a relatively high content of protein. Poultry and pig diets can be characterised as indicated in Table B of WO 01/58275, columns 2-3. Fish diets can be characterised as indicated in column 4 of this Table B. Furthermore such fish diets usually have a crude fat content of 200-310 g/kg.

WO 01/58275 corresponds to U.S. Pat. No. 6,960,462 which is hereby incorporated by reference.

An animal feed composition according to the invention has a crude protein content of 50-800 g/kg (preferably 50-600 g/kg, more preferably 60-500 g/kg, even more preferably 70-500, and most preferably 80-400 g/kg) and furthermore comprises at least one xylanase as claimed herein. In additional preferred embodiments, the crude protein content is 150-800, 160-800, 170-800, 180-800, 190-800, or 200-800 - all in g/kg (dry matter).

Furthermore, or in the alternative (to the crude protein content indicated above), the animal feed composition of the invention has a content of metabolisable energy of 10-30 MJ/kg; and/or a content of calcium of 0.1-200 g/kg; and/or a content of available phosphorus of 0.1-200 g/kg; and/or a content of methionine of 0.1-100 g/kg; and/or a content of methionine plus cysteine of 0.1-150 g/kg; and/or a content of lysine of 0.5-50 g/kg.

In particular embodiments, the content of metabolisable energy, crude protein, calcium, phosphorus, methionine, methionine plus cysteine, and/or lysine is within any one of ranges 2, 3, 4 or 5 in Table B of WO 01/58275 (R. 2-5).

Crude protein is calculated as nitrogen (N) multiplied by a factor 6.25, i.e. Crude protein (g/kg)=N (g/kg)×6.25. The nitrogen content is determined by the Kjeldahl method (A.O.A.C., 1984, Official Methods of Analysis 14th ed., Association of Official Analytical Chemists, Washington D.C.).

Metabolisable energy can be calculated on the basis of the NRC publication Nutrient requirements in swine, ninth revised edition 1988, subcommittee on swine nutrition, committee on animal nutrition, board of agriculture, national research council. National Academy Press, Washington, D.C., pp. 2-6, and the European Table of Energy Values for Poultry Feed-stuffs, Spelderholt centre for poultry research and extension, 7361 D A Beekbergen, The Netherlands. Grafisch bedrijf Ponsen & looijen by, Wageningen. ISBN 90-71463-12-5.

The dietary content of calcium, available phosphorus and amino acids in complete animal diets is calculated on the basis of feed tables such as Veevoedertabel 1997, gegevens over chemische samenstelling, verteerbaarheid en voederwaarde van voedermiddelen, Central Veevoederbureau, Runderweg 6, 8219 pk Lelystad. ISBN 90-72839-13-7.

In particular embodiments, the animal feed composition of the invention contains 0-80% maize; and/or 0-80% sorghum; and/or 0-70% wheat; and/or 0-70% Barley; and/or 0-30% oats; and/or 0-40% soybean meal; and/or 0-25% fish meal; and/or 0-25% meat and bone meal; and/or 0-20% whey.

Animal diets can e.g. be manufactured as mash feed (non-pelleted) or pelleted feed. Typically, the milled feed-stuffs are mixed and sufficient amounts of essential vitamins and minerals are added according to the specifications for the species in question. Enzymes can be added as solid or liquid enzyme formulations. For example, a solid enzyme formulation is typically added before or during the mixing step; and a liquid enzyme preparation is typically added after the pelleting step. The enzyme may also be incorporated in a feed additive or premix, as described above.

### Additional Particular Embodiments

These are additional particular embodiments of the invention:

The use in animal feed of a xylanase having a molecular weight by SDS-PAGE below 24 kDa, wherein preferably the xylanase has a degree of identity to amino acids 1-182 of SEQ ID NO: 2 of at least 50%. The xylanase may also have a percentage of identity to any one of amino acids 1-182 of SEQ ID NO: 2, amino acids 1-184 of SEQ ID NO: 4, amino acids 1-188 of SEQ ID NO: 6, or amino acids 1-228 of SEQ ID NO: 8 of at least 50%. In particular embodiments, the degree of identity is at least 55%, 60%, 65%, 70%, 75%, 80%, 85%, 90%, 95%, 97%, or at least 99%. The xylanase may be a bacterial xylanase, preferably obtainable from a bacterial strain of the genus Paenibacillus, or a variant, or fragment thereof.

The invention furthermore relates to the use of such xylanase in the preparation of a composition for use in animal feed, as well as compositions comprising such xylanase and (a) at least one fat soluble vitamin, (b) at least one water soluble vitamin, and/or (c) at least one trace mineral.

The invention also relates to an animal feed composition having a crude protein content of 50 to 800 g/kg and comprising such xylanase, as well as a method for improving the nutritional value of an animal feed, wherein such xylanase is added to the feed.

The invention also relates to the use of a xylanase as defined above in the preparation of a composition for use in animal feed.

The invention also relates to a composition comprising a xylanase as defined above, and (a) at least one fat soluble vitamin; (b) at least one water soluble vitamin; and/or (c) at least one trace mineral. The composition preferably further comprises at least one enzyme selected from the following group of enzymes: another xylanase, and/or beta-glucanase. The composition is preferably an animal feed additive.

The invention also relates to an animal feed composition having a crude protein content of 50 to 800 g/kg and comprising a xylanase as defined above.

The invention also relates to a method for improving the nutritional value of an animal feed, wherein a xylanase as defined above, or a composition as defined above, is added to the feed.

The invention also relates to the use of a xylanase as defined above for pre-treatment of animal feed or animal feed components.

### Molecular Weight

The xylanase of the invention may have a MW below 24 kDa. In particular embodiments, the MW is below 23, 22, 21, 20, 19, 18, 17, 16, 15, 14, 13, 12, 11, or below 10 kDa. In alternative embodiments, the MW is below 30, 29, 28, 27, 26, or 25 kDa.

The xylanase of the invention may also have a MW below 24000 Da. In particular embodiments, the MW is below 23000, 22000, 21000, 20000, 19000, 18000, 17000, 16000, 15000, 14000, 13000, 12000, 11000, or below 10000 Da. In alternative embodiments, the MW is below 30000, 29000, 28000, 27000, 26000, or 25000 Da.

In a particular embodiment, the indicated MW of the xylanase of the invention includes glycosylation, if any. In the alternative, the MW of the xylanase of the invention excludes glycosylation.

The MW may be determined by SDS-PAGE (Sodium Dodecyl Sulphate Poly Acryl Amide Gel Electrophoresis), which is a useful method, well known in the art, of determining molecular weight (MW) of proteins.

A suitable protocol for determining MW by SDS-PAGE is found in Example 3. In alternative embodiments, the Example 3 experiment is performed: (i) with a 10% Bis-Tris gel with MOPS running buffer; (ii) using the BenchMark Ladder (cat. no. 10747-012) commercially available from Invitrogen/Novex and which includes proteins at 20, 25 and 30kDa; and/or (iii) with Tricine and Tris-Glycine gels also available from Invitrogen/Novex. Example 3 is an SDS-PAGE of a fermentation supernatant, and there is no doubt which band represents the xylanase (only one major band; of the expected size). But in case there would be doubt, the xylanase might have to be purified to a higher extent, or, if you had an antibody you could do a western blot, or you could excise the bands in question and have an N-terminal sequence determined and this could identify the xylanase.

In the alternative, the MW of the xylanase may be calculated as the sum of the atomic masses of all the atoms of one molecule of the xylanase. To this end the average isotopic masses of amino acids in the mature protein and the average isotopic mass of one water molecule are used. The molecular weights may be derived from the 1997 IUPAC standard atomic weights. Programs for calculating MW of proteins are available, e.g. at the following internet site: http://www.expasy.org/tools/pi_tool.html (see also Gasteiger et al, in John M. Walker (ed): The Proteomics Protocols Handbook, Humana Press (2005), pp. 571-607).

In a still further alternative, the MW of the xylanase may be measured using mass spectrometry, e.g. Maldi-TOF, as is also well-known in the art.

Glycosylation is a phenomenon in which saccharides are attached to proteins. Glycosylation is only observed when expressing proteins in eukaryotes such as fungi and transgenic plants, but not in prokaryotes such as bacteria. There are various types of glycosylation: The N-linked glycosylation to the amide nitrogen of asparagine side chains, and the O-linked glycosylation to the hydroxy oxygen of serine and threonine side chains. E.g., mature glycoproteins may contain a variety of oligomannose N-linked oligosaccharides containing between 5 and 9 mannose residues.

Obviously, when a molecular weight is calculated on the basis of the protein sequence, it does not account for the effects of post-translational modifications such as glycosylation.

But glycosylation does effect protein migration in an SDS-PAGE gel, and it is also observed by mass spectrometry (Maldi-TOF). Therefore, if one wants to exclude the effect of glycosylation in these methods, the xylanase may be first deglycosylated. Deglycosylation kits are well known in the art and useful for this purpose, e.g. the Enzymatic CarboRelease™ Kit (cat. no. KE-DG01, which is commercially available from QA-Bio, LLC, 73 Sutton Place West, Palm Desert, Calif. 92211, US). This kit includes the enzymes, controls, and reagents required to remove all N-linked oligosaccharides and many O-linked sugars. The following deglycosylation enzymes are included in the kit: PNGase F (Chryseobacterium meningosepticum), O-Glycosidase (Streptococcus pneumoniae), Sialidase (Arthrobacter ureafaciens), beta-Galacto-sidase (Streptococcus pneumoniae), Glucosaminidase (Streptococcus pneumonia).

The molecular weight of a protein of course depends on the number as well as the exact chemical composition of its constituent amino acids. As an approximation of the MW, one may choose to refer only to the number of amino acids. Accordingly, the xylanase of the invention, instead of having a limitation on its molecular weight may have a mature amino acid sequence consisting of below 220 amino acid residues in. In particular embodiments of this aspect, the number of amino acids is below 215, 210, 200, or below 195; preferably below 194, 193, 192, 191, or below 190; even more preferably below 189, 188, 187, 186, 185, 184, or below 183.

In particular embodiments, (i) the xylanase of the invention is used as the sole xylanase; (ii) the xylanase is not a 23 kDa GH11 xynA from Bacillus subtilis; (iii) the xylanase is not a mature part of the xylanase having the sequence of SWISSPROT:P18429; (iv) the xylanase is not a xylanase contained in the product Belfeed B 1100 MP or ML (commercially available from BelFeed, Belgium, see: http://www.agrimex.be).

The present invention is further described by the following examples which should not be construed as limiting the scope of the invention.

### EXAMPLES

Chemicals used as buffers and substrates were commercial products of at least reagent grade.

### Example 1

### In Vitro Test of Xylanases—Solubilization of NSP

The purpose of the current study was to investigate the efficacy of various xylanases as regards solubilization of non-starch polysaccharides (NSP).

### Xylanases

The following xylanases were tested:

The RONOZYME WX xylanase, a known monocomponent animal feed xylanase derived from Thermomyces lanuginosus and commercially available from DSM Nutritional Products, Wurmisweg 576, CH-4303 Kaiseraugst, Switzerland (this xylanase is also described in WO 96/23062);

A xylanase from Paenibacillus pabuli having the amino acid sequence of amino acids 1-182 of SEQ ID NO: 2 and described in WO 2005/079585;

A xylanase from Paenibacillus sp. (polymyxa) having the amino acid sequence of amino acids 1-184 of SEQ ID NO: 4 (UNIPROT:Q9F9B9);

A xylanase (“xyl II”) from Aspergillus niger having the amino acid sequence of amino acids 1-188 of SEQ ID NO: 6 (very similar to the xylanase having SEQ ID NO: 6 in WO 97/13853); and

The mature part (excluding signal peptide and propeptide, if any) of another xylanase (“xyl III”) from Aspergillus niger, the complete amino acid sequence of which is SEQ ID NO: 8 herein (identical to the xylanase having SEQ ID NO: 9 in WO 2004/018662).

The two bacterial xylanases were expressed in Bacillus subtilis, and the two Aspergillus xylanases were expressed in Aspergillus oryzae as is known in the art. The expression strains were fermented and the xylanase-containing supernatants used in the following experiments, except for the Paenibacillus pabuli xylanase which had been further purified using standard procedures. The enzyme protein content of the xylanase supernatants was estimated based on SDS-gels, whereas the enzyme protein content of the purified Paenibacillus pabuli xylanase was determined as described below.

The study was focused on quantification of the insoluble arabinoxylan content after in vitro incubation in a procedure mimicking the gastric and small intestinal digestion steps in monogastric digestion. In the in vitro system up to 60 test tubes, containing a substrate of interest, were incubated with HCl/pepsin (simulating gastric digestion), and subsequently with pancreatin (simulating intestinal digestion). Three test tubes were used for each treatment included. At the end of the intestinal incubation phase samples of the in vitro digesta were removed and analysed for insoluble NSP.

An outline of the in vitro procedure is shown in the below diagram in which pH and temperature indicate the respective set points (target values).

### Outline of In Vitro Digestion Procedure

| | | Temper- | | Simulated |
|---|---|---|---|---|
| Components added | pH | ature | Time course | digestion phase |
| 0.8 g substrate, 4.1 ml | 3.0 | 40° C. | t = 0 min | Mixing |
| HCl (0.072M) | | | | |
| 0.5 ml HCl (0.072M)/ | 3.0 | 40° C. | t = 30 min | Gastric |
| pepsin (3000 U/g | | | | digestion |
| substrate), 0.1 ml | | | | |
| enzyme solution | | | | |
| 0.9 ml NaOH (0.215M) | 6.8 | 40° C. | t = 1.5 hours | Intestinal |
| | | | | digestion |
| 0.4 ml NaHCO₃ (1M)/ | 6.8 | 40° C. | t = 2.0 hours | Intestinal |
| pancreatin (8 mg/g diet) | | | | digestion |
| Terminate incubation | 6.8 | 40° C. | t = 6.0 hours | |

### Conditions

Substrate: 0.35 g wheat, 0.21 g barley, 0.13 g soy bean meal, and 0.11 g wheat bran, provided as a premixed diet, which was milled to pass a 0.5 mm screen
pH: stomach step=pH 3.0/intestinal step=pH 6.8-7.0
HCl: 0.072 M for 1.5 hours (i.e. 30 min HCl-substrate premixing)
pepsin: 3000 U /g diet for 1 hour (Sigma P-7000)
pancreatin: 8 mg/g diet for 4 hours (Sigma P-7545)
Temperature: 40° C.
Replicates: 3
Solutions
0.215 M NaOH
0.072 M HCl
0.072 M HCl containing 6000 U pepsin per 5 ml
1 M NaHCO₃ containing 16 mg pancreatin per ml 100 mM NaAc-buffer, pH 5.0

### Enzyme Protein Determinations

The amount of xylanase enzyme protein (EP) is calculated on the basis of the A₂₈₀ values and the amino acid sequences (amino acid compositions) using the principles outlined in S. C. Gill & P. H. von Hippel, Analytical Biochemistry 182: 319-326 (1989).

### Experimental Procedure for In Vitro Model

The experimental procedure was according to the above outline. pH was measured at time 1, 2.5, and 5.5 hours. Incubations were terminated after 6 hours and samples were removed and placed on ice before centrifugation (10000×g, 10 min, 4° C.). Supernatants were discarded and the pellet residue washed once with 100 mM acetate buffer (pH 5.0).

### Analysis

The analysis of residual NSP was made according to Theander et al., 1995, “Total dietary fiber determined as neutral sugar residues, uronic acid residues, and Klason lignin (the Uppsala method): Collaborative study”, J. AOAC Int. 7(4): 1030-1044, except that cellulose was not analysed in the present example. In brief, the starch in the sample is removed by an enzyme digestion procedure with alpha-amylase and amyloglucosidase. The non-starch polysaccharides are then precipitated with 80% ethanol and hydrolysed at 125° C. in 0.4 M sulphuric acid. Released neutral sugars are quantified by gas-liquid chromatography as alditol acetates, and their content calculated relative to an internal standard and taking the original sample weight into account.

Table 1 below shows the content (% of dry matter) of arabinose, xylose and arabinoxylan (sum of arabinose and xylose) residues in the feed after the in vitro incubation with the various xylanases. The control is without added xylanase.

TABLE 1

| | Enzyme Dosage (mg EP/kg diet) | | | | | |
|---|---|---|---|---|---|---|
| | 0 | 12.5 | 12.5 | 12.5 | 12.5 | 12.5 |
| | Xylanase Sample | | | | | |
| | | RONOZYME | | | A. niger | A. niger |
| | Control | WX | P. pabuli | P. polymyxa | xyl II | xyl III |
| Arabinose | 2.67^{a} | 2.32^{bd} | 2.11^{bc} | 2.18^{bcd} | 2.35^{d} | 2.68^{a} |
| residues | | | | | | |
| Standard | 0.20 | 0.11 | 1.04 | 0.11 | 0.15 | 0.08 |
| deviation | | | | | | |
| Relative | 100 | 87 | 79 | 82 | 88 | 100 |
| reduction | | | | | | |
| Xylose | 4.54^{a} | 3.48^{b} | 2.93^{c} | 2.98^{c} | 3.82^{d} | 4.56^{a} |
| residues | | | | | | |
| Standard | 0.27 | 0.16 | 0.05 | 0.14 | 0.22 | 0.20 |
| deviation | | | | | | |
| Relative | 100 | 77 | 65 | 66 | 84 | 100 |
| reduction | | | | | | |
| Insoluble | 7.21^{a} | 5.80^{b} | 5.04^{c} | 5.16^{c} | 6.16^{bd} | 7.25^{a} |
| Arabinoxylan | | | | | | |
| Standard | 0.46 | 0.27 | 0.09 | 0.25 | 0.38 | 0.28 |
| deviation | | | | | | |
| Relative | 100 | 80 | 70 | 72 | 85 | 100 |
| reduction | | | | | | |

^{abcd}Means within a row not sharing a common letter superscript differ with statistical significance (P < 0.05).

It appears from Table 1 that, surprisingly, the P. pabuli and P. polymyxa xylanases are statistically significantly better when it comes to solubilization of insoluble fibre polysaccharides (NSP) as compared to 1) the control without added xylanase, 2) the known animal feed xylanase of RONOZYME WX, as well as 3) the two A. niger xylanases.

### Example 2

### Dose Response Effect

The Paenibacillus pabuli xylanase was tested in various dosages in an in vitro experiment as described in Example 1.

Table 2 below shows the content (% of fresh weight) of insoluble arabinose, xylose and arabinoxylan (sum of arabinose and xylose) residues in the feed after the in vitro incubation with this xylanase in various dosages. The control is without added xylanase.

TABLE 2

| Sample | |
|---|---|
| Control | P. pabuli xylanase |

Enzyme dosage (mg EP/kg diet)

| | 0 | 0.7 | 7.0 | 70 |
|---|---|---|---|---|
| Arabinose residues | 1.99^{a} | 1.91^{ab} | 1.82^{b} | 1.68^{c} |
| Standard deviation | 0.018 | 0.043 | 0.084 | 0.053 |
| Relative reduction | 100 | 96 | 91 | 84 |
| Xylose residues | 3.26^{a} | 3.00^{b} | 2.64^{c} | 2.05^{d} |
| Standard deviation | 0.065 | 0.062 | 0.144 | 0.066 |
| Relative reduction | 100 | 92 | 81 | 63 |
| Arabinoxylan | 5.25^{a} | 4.91^{b} | 4.46^{c} | 3.73^{d} |
| Standard deviation | 0.082 | 0.104 | 0.228 | 0.119 |
| Relative reduction | 100 | 94 | 85 | 71 |

^{abcd}Means within a row not sharing a common letter superscript differ with statistical significance (P < 0.05).

It appears from Table 2 that, there is a clear and statistically significant dose-response effect of the Paenibacillus pabuli xylanase on solubilization of insoluble fibre polysaccharides (NSP).

### Example 3

### Determination of Molecular Weight

A transformed Aspergillus oryzae host expressing the xylanase of amino acids 1-188 of SEQ ID NO: 6 was fermented for four days in 500 ml baffled shake flasks with 100 ml YP+2% G medium (10 g yeast extract, 20 g peptone, water to 1 L, autoclave at 121° C., 20 minutes, add 100 ml 20% sterile glucose solution) at 30° C. and 200 RPM. The fermentation liquor was filtered through a 0.22 um (micrometer) filter unit to provide a supernatant.

10 ul (microliter) of the supernatant was mixed with 10 ul NuPAGE® LDS sample buffer (4×) (available from Invitrogen, cat. no. NP0007), 2 ul 1% EDTA, 2 ul 6% PMSF, 4 ul 0.5 M DTT, and 2 ul H₂O, to a total volume of 20 ul.

The 20 ul sample was heated to 99° C. for 3 minutes and applied to an SDS-PAGE gel of the type NuPAGE® Novex 10% Bis-Tris 1 mm Gels, available from Invitrogen (cat. no. NP0301BOX).

Running buffer: Upper buffer chamber, 200 ml 1× NuPAGE® MES SDS running buffer (cat. no. NP0002) containing 500 ul NuPAGE® antioxidant (cat. no. NP0005). Lower buffer chamber, 600 ml 1× NuPAGE® MES SDS running buffer.

Run conditions: Two steps, viz. 30 min 50 mAmp, and 20 min 100 mAmp.

Stain: Simply Blue Stain™ Safe stain from Invitrogen (cat. no. LC6065).

Rinse: 3 times for 5 min in de-ionizied water, approximately 100 ml for each time.

Stain: Cover the gel with Simply Blue stain solution. Stain for at least one hour at room temperature.

Destain: Discard the stain and wash the gel in deionizied water.

MW marker: Amersham's Low Molecular Weight Calibration Kit for SDS Electrophoresis (product code 17-0446-01).

From the SDS-PAGE gel, the molecular weight of the xyl II Aspergillus niger xylanase was judged to below 24 kDa.

### Example 4

### Determination of Xylanase Activity

This assay is an example of a xylanase assay. It is particularly suitable for determining the activity of the Paenibacillus xylanases of the present invention.

Substrate: 0.2% AZCL-Arabinoxylan from wheat (Megazyme) in 0.2 M Na-phosphate buffer pH 6.0+0.01% Triton-x-100.

Standard: Bio-Feed Wheat FXU standard (such as batch 43-1195, which is available on request from Novozymes NS, Krogshoejvej 36, DK-2880 Bagsvaerd, Denmark).

Dilution: In 0.01% Triton-x-100.

FXU/ml: 0.05; 0.10; 0.15; 0.20; 0.25; 0.30; 0.40.

Method: 900 ul (microliter) substrate is preheated to 37° C. in a thermomixer. 100 ul sample is added. Incubate for 15 min at 37° C. at maximum speed. On ice for 2 min. Spin 1 min 20000×G. 2×200 ul supernatant is transferred to a micro titter plate. Endpoint OD 590 nm is measured.

### Example 5

### Solubilization and Total Degradation of NSP in Vitro

The purpose of the current study was to compare the efficacy of a xylanase of the invention with a homologous, known animal feed xylanase as regards solubilization and total degradation of non-starch polysaccharides (NSP).

### Xylanases

The following xylanases were tested:

The RONOZYME WX xylanase and a xylanase of the invention from Paenibacillus pabuli (both described in Example 1), and a comparative xylanase having the sequence of amino acids 1-185 of SEQ ID NO: 9 (Swissprot Q6TLP3).

The comparative xylanase was expressed from a synthetic gene in a protease-weak strain of Bacillus subtilis. A xylanase-containing culture broth was prepared by fermentation thereof, and the xylanase was purified using standard procedures. To inactivate possible proteases, the filtered culture broth, added the same volume of 50 mM acetic acid pH 4.0 and adjusted to pH 4.0 with 50% acetic acid, was incubated for 30 minutes at 37° C. in 500 mL shakeflasks containing 250 mL in a waterbath. The solution was mixed gently with a magnetic stirrer during the incubation. After incubation, the solution was centrifuged for 30 minutes at 12,000 g and the supernatant was separated from the pellet. The protease activity was measured as follows: Substrate N-Succinyl-Ala-Ala-Pro-Phe-pNitroanilid (Sigma, S7388), assay buffer: 100 mM HEPES pH 7.5 (0.01% Triton X-100), enzyme dilutions in 0.01% Triton X-100, and reading OD405 after 10 minutes at 25° C.

The resulting xylanase was substantially pure (one band on an SDS-gel) and had a moleular weight of approximately 22 kDa (by SDS-PAGE).

The enzyme protein content of the commercial xylanase was estimated based on SDS-gels, whereas the enzyme protein content of the purified Paenibacillus pabuli xylanase and the comparative xylanase was determined as described in Example 1.

### Experimental Procedure for In Vitro Model

The study was focused on quantification of insoluble as well as total arabinoxylan content after in vitro incubation in a monogastric digestion procedure as described in Example 1 (See: Outline of in vitro digestion procedure, Conditions, Solutions, and Enzyme protein determinations).

For determination of insoluble arabinoxylans, pH was measured at time 1, 2.5, and 5.5 hours. Incubations were terminated after 6 hours and samples were removed and placed on ice before centrifugation (10000×g, 10 min, 4° C.). Supernatants were discarded and the insoluble pellet residue was washed once with acetate buffer (pH 5.0 and 100 mM).

For determination of total arabinoxylans, pH was measured at time 1, 2.5, and 5.5 hours. Incubations were terminated after 6 hours. Absolute ethanol was added to obtain a concentration of 80% ethanol in the sample in order to precipitate all polysaccharides of a degree of polymerization (DP) greater than 10 (DP>10). Samples were then cooled (4° C.) on ice before centrifugation (10000×g, 10 min, 4° C.). Supernatants were discarded and the pellet residue washed once with 80% ethanol.

### Analysis

The analysis of arabinoxylan NSP in the pellet residue was performed according to Theander et al, as described in Example 1. Polysaccharides (DP>10) are hydrolysed in sulphuric acid together with an internal standard (Myo-inositol) and released neutral sugars (arabinose+xylose) quantified.

### Results

Table 3 shows the dry matter content (%) of insoluble arabinose+xylose (arabinoxylan) residues in the feed, i.e. arabinoxylan NSP which is insoluble after the in vitro incubation with the xylanases.

Table 4 shows the dry matter content (%) of total arabinose+xylose (arabinoxylan) residues in the feed, i.e. the sum of insoluble and soluble arabinoxylan NSP.

In both Tables the control is without added xylanase.

TABLE 3

| | Enzyme Dosage (mg EP/kg diet) | | | | | |
|---|---|---|---|---|---|---|
| | 0 | 5 | 5 | 20 | 5 | 20 |
| | Xylanase Sample | | | | | |
| | | RONOZYME | | | | |
| | Control | WX | P. pabuli | P. pabuli | Q6TLP3 | Q6TLP3 |
| Insoluble | 7.34^{a} | 6.89^{b} | 5.78^{de} | 5.51^{e} | 6.36^{c} | 6.00^{cd} |
| Arabinoxylan | | | | | | |
| Standard | 0.42 | 0.076 | 0.22 | 0.20 | 0.05 | 0.14 |
| deviation | | | | | | |
| Relative | 100 | 94 | 79 | 75 | 87 | 82 |
| reduction | | | | | | |

^{abcde}Means within a row not sharing a common letter superscript differ with statistical significance (P < 0.05).

TABLE 4

| | Enzyme Dosage (mg EP/kg diet) | | | | | |
|---|---|---|---|---|---|---|
| | 0 | 5 | 5 | 20 | 5 | 20 |
| | Xylanase Sample | | | | | |
| | | RONOZYME | | | | |
| | Control | WX | P. pabuli | P. pabuli | Q6TLP3 | Q6TLP3 |
| Total | 7.94^{ab} | 8.17^{a} | 7.48^{cd} | 7.32^{d} | 7.82^{abc} | 7.57^{bcd} |
| Arabinoxylan | | | | | | |
| Standard | 0.16 | 0.26 | 0.072 | 0.25 | 0.31 | 0.11 |
| deviation | | | | | | |
| Relative | 100 | 103 | 94 | 92 | 98 | 95 |
| reduction | | | | | | |

^{abcd}Means within a row not sharing a common letter superscript differ with statistical significance (P < 0.05).

It appears from Table 3 that, surprisingly, the P. pabuli xylanase is statistically significantly better regarding the capacity to solubilize the arabinoxylan fraction as compared to 1) the control without added xylanase, 2) the commercial animal feed xylanase of RONOZYME WX, as well as 3) the comparative Q6TLP3 xylanase.

The content of soluble arabinoxylans will go into the supernatant after centrifugation of the in vitro incubation mixtures, and will therefore not be included in the determination of insoluble arabinoxylans.

The content of total arabinoxylan (Table 4) includes the content of insoluble arabinoxylans as well as the content of soluble arabinoxylans.

The differences between corresponding Table 4 and Table 3 values are indicative of the amount of NSP which has been degraded to oligomers smaller than DP 10 by the xylanases during the in vitro incubation.

Clearly, the xylanases investigated are more efficient in the solubilization of the arabinoxylan fraction (Table 3) than they are in the total degradation (Table 4). This is a typical trait of family 11 xylanases. Still it appears from Table 4 that the P. pabuli xylanase at 5 mg EP/kg diet is also significantly better regarding the capacity to degrade the arabinoxylan fraction as compared to 1) the control without added xylanase, and 2) the known animal feed xylanase of RONOZYME WX, and it is 3) numerically more efficient (4%) than the comparative Q6TLP3 xylanase.

### Example 6

### Animal Feed and Feed Additive Compositions

A formulation of the Paenibacillus pabuli xylanase of SEQ ID NO: 2 containing 0.050 g xylanase enzyme protein is added to the following premix (per kilo of premix):

| | | |
|---|---|---|
| 5000000 | IE | Vitamin A |
| 1000000 | IE | Vitamin D3 |
| 13333 | mg | Vitamin E |
| 1000 | mg | Vitamin K3 |
| 750 | mg | Vitamin B1 |
| 2500 | mg | Vitamin B2 |
| 1500 | mg | Vitamin B6 |
| 7666 | mcg | Vitamin B12 |
| 12333 | mg | Niacin |
| 33333 | mcg | Biotin |
| 300 | mg | Folic Acid |
| 3000 | mg | Ca-D-Panthothenate |
| 1666 | mg | Cu |
| 16666 | mg | Fe |
| 16666 | mg | Zn |
| 23333 | mg | Mn |
| 133 | mg | Co |
| 66 | mg | I |
| 66 | mg | Se |

| | |
|---|---|
| 5.8% | Calcium |
| 25% | Sodium |

### Animal Feed

This is an example of an animal feed (broiler feed) comprising 0.5 mg/kg (0.5 ppm) of the Paenibacillus pabuli xylanase of SEQ ID NO: 2 (calculated as xylanase enzyme protein):

65.00% wheat
32.35% Soybean meal (50% crude protein, CP)
1.0% Soybean oil
0.2% DL-Methionine
0.22% DCP (dicalcium phosphate)
0.76% CaCO₃ (calcium carbonate)
0.32% Sand
0.15% NaCl (sodium chloride)
1% of the above Premix

The ingredients are mixed, and the feed is pelleted at the desired temperature, e.g. 70° C.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

Various references are cited herein, the disclosures of which are incorporated by reference in their entireties.

1-11. (canceled)

12. An animal feed composition comprising a xylanase which has the sequence of amino acids 1-182 of SEQ ID NO: 2, and
(a) at least one fat soluble vitamin;
(b) at least one water soluble vitamin; and
(c) at least one trace mineral.

13. The composition of claim 12, which comprises a fat soluble vitamin.

14. The composition of claim 13, wherein the fat soluble vitamin is selected from the group consisting of vitamin A, vitamin D3, vitamin E, and vitamin K.

15. The composition of claim 14, wherein the fat soluble vitamin is vitamin K3.

16. The composition of claim 12, which comprises a water soluble vitamin.

17. The composition of claim 16, wherein the water soluble vitamin is selected from the group consisting of biotin, choline, folic acid, niacin, panthothenate, vitamin B1, vitamin B2, vitamin B6, and vitamin B12.

18. The composition of claim 12, which comprises a trace mineral.

19. The composition of claim 18, wherein the trace mineral is selected from the group consisting of cobalt, copper, iodine, iron, manganese, selenium, and zinc.

20. The composition of claim 12, which further comprises a cereal grain.

21. The composition of claim 20, wherein the cereal grain is selected from the group consisting of barley, maize, sorghum, and wheat.

22. The composition of claim 12, which comprises barley, soy bean meal, wheat, and wheat bran.

23. The composition of claim 12, further comprising a beta-glucanase.

24. The composition of claim 12, which is an animal feed composition having a crude protein content of 50 to 800 g/kg.

25. A method for improving the nutritional value of an animal feed, comprising adding the composition of claim 12 to the animal feed.

26. A method for the degradation or solubilization of non-starch polysaccharides during gastric and intestinal digestion, comprising administering the composition of claim 12 to an animal.


SEQUENCE LISTING (9 sequences)

SEQ ID NO: 1 - DNA, 630 bp, Paenibacillus pabuli
  CDS (1)..(630)
  sig_peptide (1)..(84)
  mat_peptide (85)..(630)
atgtttaaattcggaaaaaaattgttaactgttgtccttgccgcttccatgagttttggtgtattcgccgctacgacaggtgctacagattactggcagaactggacagatggcgggggtactgttaatgccgtgaacggttcgggaggaaactacagtgtaaactggcagaacacggggaactttgttgtcggtaaagggtggacttacggtacacctaatcgtgtagtgaattacaatgcgggtgtattctctccatccggcaacggatatttgacgttttacgggtggacacggaatgcacttattgaatactacgtggtggataactggggaacataccggccaaccggaacatacaaaggcacagtaaccagtgatggtggcacatatgacatctatactacgatgagatacaatcagccatccattgacgggtattcaacattcccgcaatactggagtgttagacaatccaaacgtccaatcggtgtaaattcccaaattacgttccagaatcacgtaaatgcgtgggcgagcaagggcatgtacttgggtaacagctggtcctatcaagtgatggccaccgaaggatatcaaagtagcggtagttcgaatgtgactgtttgg

SEQ ID NO: 2 - protein, 210 aa, Paenibacillus pabuli
MFKFGKKLLTVVLAASMSFGVFAATTGATDYWQNWTDGGGTVNAVNGSGGNYSVNWQNTGNFVVGKGWTYGTPNRVVNYNAGVFSPSGNGYLTFYGWTRNALIEYYVVDNWGTYRPTGTYKGTVTSDGGTYDIYTTMRYNQPSIDGYSTFPQYWSVRQSKRPIGVNSQITFQNHVNAWASKGMYLGNSWSYQVMATEGYQSSGSSNVTVW

SEQ ID NO: 3 - DNA, 1409 bp, Unknown
  Paenibacillus species
attacccgtaataaaatggtaatccagtaatatcagtccagttggccgagctcatcttgattcaattttaactaattggcaagcattgagtgacacatgaaatccgggtattttgtcttcagattctctctcatttcttatctctttcgaacaataacacttttgttgatccagttaactgtgtcttcatgaaacgtgtgatagcacctgtacctgacccgagctctgcgacggcctttacctcaccccagggtacagattcaaccaccttagctgccagaaatcgagaactaggcataacgcttcccactcgttgtggattggtgaggaatccttgcaaaaaaagtaggtattctttgaaattcaaccgtttatcccccatgacttattttcttccttttgttttttgttcatataaaaaacagcagcgaaaaaatctaaggattttacgaggatatatctaaagaaaatctgaagaactctaaagaagaaactcaataaactcccgacggatatatataacttagggaaatctaaacatagtaaagtgtaatatttaatttgcaaagtggttatgtgctcgttattattatataattttacatataaaggaggtgaaagtaagaaaacacgggatatggttcacatgagtgatgcactgcaacggagaatcttccgacaagcaggtgtagaacgaaaatcacaaaattttaggaggcaaattatgtttaaaagtagtaagaaactgttaacggtagttcttgcagcttccatgagttttggtttttttgcatcaacctcaaatgcagcgacggactactggcaaaattggaccgatggcggtgggacggttaatgctgttaatgggtccggcggcaattacagcgtaacatggaaaaatagcgggaattttgttgtcggcaaaggctggactactggatcgccagacagaacgattaattacaatgccggtgtctgggcgccgtccggtaatggatatttggccctctacgggtggacgagaaactcactcatcgaatattacgttgttgatagctgggggacttatcgacctaccggaacgtataaaggtacggtgaccagtgatgggggtacatatgacatctacacaacaatgcgatacgacgcaccttccattgaaggccaaaaaacgacatttatccagtactggagtgttcgacagacgaagagaccgaccgggggcaactccacgatcactttcagcaatcacgtgaaggcttgggcgaggcaaggaatgcatctggggaacaactggtcttaccaggtgttagcgacagaggggtatcagagtagcgggagctctaacgtaacggtgtggtaaccatgaaggcttaaagccgtattacaacatagataaggaaaagaaa

SEQ ID NO: 4 - protein, 212 aa, Unknown
  Synthetic Construct
MFKSSKKLLTVVLAASMSFGFFASTSNAATDYWQNWTDGGGTVNAVNGSGGNYSVTWKNSGNFVVGKGWTTGSPDRTINYNAGVWAPSGNGYLALYGWTRNSLIEYYVVDSWGTYRPTGTYKGTVTSDGGTYDIYTTMRYDAPSIEGQKTTFIQYWSVRQTKRPTGGNSTITFSNHVKAWARQGMHLGNNWSYQVLATEGYQSSGSSNVTVW

SEQ ID NO: 5 - DNA, 786 bp, Aspergillus niger
  CDS (6)..(680)
  sig_peptide (6)..(116)
  mat_peptide (117)..(680)
caatcatgctcaccaagaaccttctcctctgctttgccgcggctaaggctgctctggctgttccccacgactctgtcgcccagcgttcggatgccttgcacatgctctctgagcgctcgaccccgagctcgaccggcgagaacaacggcttctactactccttctggaccgacggcggtggcgacgtgacctacaccaacggagatgctggtgcctacactgttgagtggtccaacgtgggcaactttgtcggtggaaagggctggaaccccggaagtgcgcaggacatcacctacagcggcaccttcacccctagcggcaacggctatctctccgtctatggctggaccactgaccccctgatcgagtactacatcgtcgagtcctacggcgactacaaccccggcagtggaggcacatacaagggcaccgtcacctcggacggatccgtttacgatatctacacggctacccgtaccaatgctgcttccattcagggaaccgctaccttcactcagtactggtccgtccgccagaacaagagagttggcggaactgttaccacctccaaccacttcaatgcttgggctaagctgggaatgaacctgggtactcacaactaccagatcgtggctaccgagggttaccagagcagtggatcttcgtccatcactgttcagtaagcggtggatgtgtggattgaacgattgtgcatgtaaatactgagcagtcgtatgatatgtgaaacaggtagttgtttggtaccaatgtactggtcatttggag

SEQ ID NO: 6 - protein, 225 aa, Aspergillus niger
MLTKNLLLCFAAAKAALAVPHDSVAQRSDALHMLSERSTPSSTGENNGFYYSFWTDGGGDVTYTNGDAGAYTVEWSNVGNFVGGKGWNPGSAQDITYSGTFTPSGNGYLSVYGWTTDPLIEYYIVESYGDYNPGSGGTYKGTVTSDGSVYDIYTATRTNAASIQGTATFTQYWSVRQNKRVGGTVTTSNHFNAWAKLGMNLGTHNYQIVATEGYQSSGSSSITVQ

SEQ ID NO: 7 - DNA, 1086 bp, Aspergillus niger
  CDS (65)..(832)
  sig_peptide (65)..(148)
  mat_peptide (149)..(832)
caccctcaactttcaagtcacagttgtacccttctacttggatatattctttcttatcatcatcatggtgtctttccttggccaagcacgcctggccgtgccaatcctgtcagcttttgcgtgcatgcttgcggcaagttccgccatccccccaccaccgcgtggagctctttcacccgaacgtctacaatggattagagaggtgatcggcaatcagactgagaacgacagcgtttcggaccttgctaagcggagcactatcctgcataccagccaagacggagttgacagcgccggattctactactcagtatacaatgacaacggggccgatgttggatacaccgaataccccacgaccggccagttcgaacttggctggagtgctgaggcggaattcctcgctggaaagggcttcaagggcggcaacccacgctccttgacctgggacggttatttcaccgccgaaggggactggactttggccatttatggctggaccacaaaccccgtcaccgagtggtatattgtggagtcgcatggaagtggaaccccaggcaacgggaacatcctcggccaggttgatagtgatggcggcgtctacgatgtctataatctcccctatagaaatgttccggagatctacggtgtcaccaacttcgaccagcactggtcggtgcgtcgctcccaccgctctactggcaccgtcgacgtgagcgcccatttccagcgctggaaggaattgggtctcacccctggctccccagtcttccagatggttaccctggaaggcttttccggccaggggtacctggacttcaccgttagtgcatagaaaggttcagggacctcgtggtctcagctggcggcgagcacttacttgaggccagatcccctccacgtgtatctttttttctagggcaaagaatggtgacttcggttcaagaaacagaagcccccctttcccttcctttttgtttcttcttctcttagagcctgctctttaacaacttgttcgcttcttttccagttaccaattattttttgaccttcaaagatatttactatcgaactaatttgtttatc

SEQ ID NO: 8 - protein, 256 aa, Aspergillus niger
MVSFLGQARLAVPILSAFACMLAASSAIPPPPRGALSPERLQWIREVIGNQTENDSVSDLAKRSTILHTSQDGVDSAGFYYSVYNDNGADVGYTEYPTTGQFELGWSAEAEFLAGKGFKGGNPRSLTWDGYFTAEGDWTLAIYGWTTNPVTEWYIVESHGSGTPGNGNILGQVDSDGGVYDVYNLPYRNVPEIYGVTNFDQHWSVRRSHRSTGTVDVSAHFQRWKELGLTPGSPVFQMVTLEGFSGQGYLDFTVSA

SEQ ID NO: 9 - protein, 214 aa, unknown
  Bacterium from environmental sample
MFKLSKKILMVLLTISMSFISLFTVTAYAASTDYWQNWTDGGGTVNATNGSDGNYSVSWSNCGNFVVGKGWTTGSATRVINYNAGAFSPSGNGYLALYGWTRNSLIEYYVVDSWGTYRPTGTYKGTVTSDGGTYDIYTTTRTNAPSIDGNNTTFTQFWSVRQSKRPIGTNNTITFSNHVNAWKSKGMNLGSSWSYQVLATEGYQSSGYSNVTVW